(19) United States
(12) Patent Application Publication
(10) Pub. No.: US 2008/0026016 A1
(43) Pub. Date: Jan. 31, 2008

(54) Aqueous Dispersions of Low-Water Solubility or Water-Insoluble Active Substances and Dry Powders Produced Therefrom

(75) Inventors: Christian Koepsel, Weinheim (DE); Jesper Feldthusen Jensen, Mainz (DE); Klaus Kramer, Landau (DE)

Correspondence Address: CONNOLLY BOVE LODGE & HUTZ, LLP, P O BOX 2207, WILMINGTON, DE, 19899, US

(73) Assignee: BASF Aktiengesellschaft, Ludwigshafen (DE)

(21) Appl. No.: 11/630,457

(22) PCT Filed: Jun. 17, 2005
(86) PCT No.: PCT/EP05/06547

§ 371 Date: Dec. 21, 2006

(30) Foreign Application Priority Data
Jun. 23, 2004 (DE) 10 2004 030 072.0

(51) Int. Cl. A61K 9/10; A61K 9/14

(52) U.S. Cl. 424/401; 424/439; 424/442; 424/489; 426/540

(57) ABSTRACT

The present invention relates to aqueous dispersions and dry powders prepared therefrom, comprising at least one active substance which is sparingly soluble, or insoluble, in water and rice starch as protective colloid.

The present invention relates to aqueous dispersions comprising at least one active substance which is sparingly soluble, or insoluble, in water, and rice starch as protective colloid.

A number of active substances which are suitable for the food-and-feed sector or for pharmaceutical and cosmetic applications, for example fat-soluble vitamins, carotenoids, but also the natural colorants curcumin or carmine, and a large number of UV filters can only be employed in the form of specifically stabilized preparations as the result of the fact that they are insoluble in water, and/or as the result of their susceptibility to oxidation. The direct use of the crystalline substances for, inter alia, coloring aqueous foodstuffs, as feed additives or as active substances and agents in cosmetic preparations is, as a rule, not possible. The high requirements regarding bioavailability, coloring properties and dispersibility, in particular in aqueous media, but also in lipophilic media, can only be met by specific formulations.

When dyeing foodstuffs directly, satisfactory color yields can only be achieved by preparations in which the active substances, for example carotenoids, are present in finely divided form and protected from oxidation by protective colloids. These formulations, which are used in animal feeds, lead to a higher bioavailability of the active substances and thus indirectly to better coloring effects, for example in the pigmentation of egg yolks or fish.

A series of different formulation methods, all of which aim at reducing the crystallite size of the active substances and bringing it down to a particle size range of less than 10 μm, are already known from the literature.

A large number of methods, for example described in Chimia 21, 329 (1967), WO 91/06292 and in WO 94/19411, resort to the use of a colloid mill when grinding carotenoids, by means of which particle sizes of from 2 to 10 μm are obtained.

There also exist combined emulsifying/spray-drying methods as they are described for example in DE-A-12 11 911 or in EP-A-0 410 236.

In accordance with the European patent specification EP-B-0 065 193, finely divided pulverulent carotenoid preparations are prepared by dissolving for example β-carotene in a volatile, water-miscible organic solvent at temperatures of between 50° C. and 200° C., if appropriate under elevated pressure, within a period of less than 10 seconds.

The β-carotene is precipitated from the resulting molecularly disperse solution by instant rapid mixing with an aqueous solution of a protective colloid at temperatures of between 0° C. and 50° C. This gives a colloid-disperse β-carotene hydrosol with an orangey-yellow tint. Subsequent spray-drying of the dispersion yields a free-flowing dry powder which dissolves in water with the formation of a clear, yellowy-orange dispersion.

An analogous process for the preparation of finely divided pulverulent carotenoid preparations is described in EP-A-0 937 412, where solvents which are immiscible in water are used.

WO 98/26008 relates to the use of a mixture of low-molecular-weight and high-molecular-weight protective colloids for the preparation of redispersible xanthophyll-comprising dry powders.

It is the aim of the present invention to convert hydrophobic active substances and agents which are sparingly soluble, or insoluble, in water into stable aqueous dispersions or into stable, readily redispersible dry powders.

For the purposes of the invention, stable means that the formulations are, inter alia, stable to oxidation, the effect of light, sedimentation and creaming over a period and temperature range which is sufficient for the application in question.

It was therefore an object of the present invention to provide natural polymers which can be employed as protective colloids, in particular in cosmetics, pharmaceuticals, foodstuffs, food supplements and feedstuffs.

This object has been achieved by aqueous dispersions comprising at least one active substance which is sparingly soluble, or insoluble, in water and rice starch as protective colloid.

For the purposes of the present invention, the term aqueous dispersions is understood as meaning both aqueous suspensions and emulsions. Aqueous suspensions in which the dispersed phase comprises at least one active substance which is sparingly soluble, or insoluble, in water as nanoparticulate particles are to be mentioned by preference. In a prominent position of the invention are, in addition, the dry powders or emulsions, preferably double emulsions, in particular o/w/o emulsions, prepared from the above aqueous dispersions.

In the present context, organic active substances which are sparingly soluble in water are understood as meaning compounds whose solubility in water is <5% by weight, preferably <1% by weight, especially preferably <0.1% by weight, very especially preferably <0.01% by weight.

Examples of active substances which may be mentioned within the scope of the present invention for the food-and-feed sector and for pharmaceutical and cosmetic applications are the following compounds:

fat-soluble vitamins such as, for example, the K vitamins, vitamin A and derivatives such as vitamin A acetate, vitamin A propionate or vitamin A palmitate, vitamin D₂ and vitamin D₃, and vitamin E and derivatives. In the present context, vitamin E represents natural or synthetic α-, β-, γ- or δ-tocopherol, preferably natural or synthetic α-tocopherol, and tocotrienol. Examples of vitamin E derivatives are tocopheryl-C₁-C₂₀-carboxylic esters such as tocopheryl acetate or tocopheryl palmitate.

Polyunsaturated fatty acids such as, for example, linoleic acid, linolenic acid, arachidonic acid, eicosapentaenoic acid, docosahexaenoic acid.

Food colorants such as curcumin, carmine or chlorophyll.

Carotenoids, both carotenes and xanthophylls, such as, for example, β-carotene, lycopene, lutein, astaxanthin, zeaxanthin, capsanthin, capsorubin, cryptoxanthin, citranaxanthin, canthaxanthin, bixin, β-apo-4-carotenal, β-apo-8-carotenal and β-apo-8-carotenoic acid ethyl ester.

Phytosterols, coenzyme Q10.

Organic UV filters which are insoluble, or sparingly soluble, in water, such as, for example, compounds from the group of the triazines, anilides, benzophenones, triazoles, cinnamamides and the sulfonated benzimidazoles.

Preferred active substances are carotenoids, in particular β-carotene, lycopene, lutein, astaxanthin and canthaxanthin and vitamin E, and from the series of the UV filters, the triazine class of substances, in particular Uvinul T150.

An especially preferred embodiment of the aqueous dispersions according to the invention takes the form of aqueous suspensions comprising at least one of the active substances which are sparingly soluble, or insoluble, in water which have been mentioned at the outset as nanoparticulate particles.

Depending on the formulation method used, the mean particle size of the nano-particulate particles in the aqueous dispersion is in the range of from 0.01 to 100 μm, preferably in the range of from 0.01 to 10 μm, especially preferably in the range of from 0.01 to 2 μm, very especially preferably in the range of from 0.02 to 1 μm.

The amounts of the different components of the dispersions, in particular suspensions, according to the invention, are chosen in accordance with the invention in such a way that the preparations comprise 0.1 to 90% by weight, preferably 2 to 40% by weight, especially preferably 3 to 30% by weight, very especially preferably 5 to 25% by weight, of at least one active substance which is sparingly soluble, or insoluble, in water, and 0.1 to 99.9% by weight, preferably 5 to 70% by weight, especially preferably 10 to 60% by weight, very especially preferably 15 to 35% by weight, of rice starch. The percentages by weight are based on the dry matter of the formulation in each case.

In addition, the preparations can also comprise low-molecular-weight stabilizers such as antioxidants and/or preservatives to protect the active substances. Examples of suitable antioxidants or preservatives are α-tocopherol, ascorbic acid, tert-butyl-hydroxytoluene, tert-butylhydroxyanisole, lecithin, ethoxyquin, methylparaben, propylparaben, sorbic acid or sodium benzoate. The antioxidants or preservatives can in amounts of 0.01 to 50% by weight, preferably 0.1 to 30% by weight, especially preferably 0.5 to 20% by weight, very especially preferably 1 to 10% by weight, based on the dry matter of the formulation.

The dispersions can furthermore also comprise plasticizers for increasing the mechanical stability of any dry powder prepared therefrom. Examples of suitable plasticizers are sugars and sugar alcohols such as sucrose, glucose, lactose, invert sugar, sorbitol, mannitol, xylitol or glycerol. Lactose is preferably employed as plasticizer. The plasticizers can be present in amounts of from 0.1 to 70% by weight, preferably 10 to 60% by weight, especially preferably 20 to 50% by weight, based on the dry matter of the formulation.

The dispersions may further comprise low-molecular-weight surface-active compounds (emulsifiers) in a concentration of from 0.01 to 70% by weight, preferably 0.1 to 50% by weight, especially preferably 0.5 to 20% by weight, based on the dry matter of the formulation. Suitable such compounds are, especially, amphiphilic compounds or mixtures of such compounds. In principle, all surfactants with an HLB value of from 5 to 20 are suitable. Examples of suitable such surface-active substances are: esters of long-chain fatty acids with ascorbic acid, mono- and diglycerides of fatty acids and their ethoxylation products, esters of monofatty acid glycerides with acetic acid, citric acid, lactic acid or diacetyltartaric acid, polyglycerol fatty acid esters such as, for example, triglycerol monostearate, sorbitan fatty acid esters, propylene glycol fatty acid esters and lecithin. Ascorbyl palmitate is preferably employed.

Under certain circumstances, it may also be advantageous additionally to employ a physiologically acceptable oil such as, for example, sesame seed oil, corn oil, cottonseed oil, soya oil or peanut oil, and cosmetic oils, for example liquid paraffin, glyceryl stearate, isopropyl myristate, diisopropyl adipate, cetyl stearyl 2-ethyl-hexanoate, hydrogenated polyisobutene, Vaseline, caprylic/capric triglycerides, microcrystalline wax, lanolin and stearic acid in a concentration of from 0.1 to 500% by weight, preferably 10 to 300% by weight, especially preferably 20 to 100% by weight, based on the active substance(s) which is/are sparingly soluble, or insoluble, in water.

The invention also relates to a process for the preparation of an aqueous dispersion of at least one active substance which is sparingly soluble, or insoluble, in water, by dispersing, in an aqueous molecularly disperse or colloid-disperse solution of a protective colloid, one or more active substance(s) which is/are sparingly soluble, or insoluble, in water, which comprises using rice starch as protective colloid.

In a preferred embodiment of the process according to the invention, the dispersing step takes the form of the preparation of a suspension of at least one active substance which is sparingly soluble, or insoluble, in water, in an aqueous molecularly disperse or colloid-disperse solution of a rice starch.

In an especially preferred embodiment of the process, the dispersing step, in particular suspending, comprises:
a₁) dissolving at least one active substance which is sparingly soluble, or insoluble, in water in one or more water-miscible organic solvents or in a mixture of water and one or more water-miscible organic solvent(s), or
a₂) dissolving, in one or more organic solvent(s) which is/are immiscible with water at least one active substance which is sparingly soluble, or insoluble, in water,
b) mixing of the solution obtained in a₁) or a₂) with an aqueous molecularly disperse or colloid-disperse solution of a rice starch, the hydrophobic phase of the active substance which is sparingly soluble, or insoluble, in water being generated as a nanodisperse phase, and
c) removing the organic solvent.

The water-miscible solvents used in step a₁) are, mainly, water-miscible, thermally stable, volatile solvents which only comprise carbon, hydrogen and oxygen, such as alcohols, ethers, esters, ketones and acetals. Solvents which are expediently used are those which are water-miscible to at least 10%, have a boiling point of below 200° C. and/or have less than 10 carbons. Substances which are especially preferably used are methanol, ethanol, n-propanol, isopropanol, 1,2-butanediol 1-methyl ether, 1,2-propanediol 1-n-propyl ether, tetrahydrofuran or acetone.

In the context of the present invention, the term “an organic solvent which is immiscible with water” means an organic solvent which is soluble to less than 10% in water under atmospheric pressure. Examples of possible solvents in this context are, inter alia, halogenated aliphatic hydrocarbons such as, for example, methylene chloride, chloroform and carbon tetrachloride, carboxylic esters such as dimethyl carbonate, diethyl carbonate, propylene carbonate, ethyl formate, methyl acetate, ethyl acetate or isopropyl acetate, and ethers such as methyl tert-butyl ether. Preferred water-immiscible organic solvents are the following compounds from the group consisting of dimethyl carbonate, propylene carbonate, ethyl formate, ethyl acetate, isopropyl acetate and methyl tert-butyl ether.

A solvent which is especially preferably used for the dispersing/suspending step is at least one water-miscible, organic solvent or a mixture of water and at least one water-miscible organic solvent, very especially preferably isopropanol or acetone.

An advantageous embodiment of the abovementioned process according to the invention is characterized in that the molecularly disperse solution of at least one active substance which is sparingly soluble, or insoluble, in water is prepared in step a) at temperatures above 30° C., preferably between 50° C. and 240° C., in particular 100° C. to 200° C., especially preferably 140° C. to 180° C., if appropriate under pressure, and immediately thereafter, in step b), treated with the aqueous solution of the protective colloid, a mixing temperature of between 35° C. to 120° C. being established.

During this process, the solvent component is transferred into the aqueous phase, and the hydrophobic phase of the active substance(s) is generated as nanodisperse phase.

As regards a more detailed description of the processes and equipment for the abovementioned dispersing step, reference is made at this point to EP-B-0 065 193.

The invention furthermore relates to a process for the preparation of a dry powder comprising at least one active substance which is sparingly soluble, or insoluble, in water as nanoparticulate particles, wherein the above-described aqueous dispersions, in particular suspensions, are freed from the water and dried.

In this context, the conversion into a dry powder can be accomplished inter alia by spray-drying, spray-cooling, freeze-drying or fluidized-bed drying, if appropriate also in the presence of a coating material. Suitable coating materials are, inter alia, corn starch, silica or else tricalcium phosphate.

In a preferred embodiment of the abovementioned process, the resulting suspension of at least one active substance which is sparingly soluble, or insoluble, in water is ground before its conversion into a dry powder.

In this context, the grinding step can be accomplished in a known manner, for example using a ball mill. Depending on the type of mill used, the mixture is ground until the particles show a mean particle size D[4.3] of from 0.1 to 100 μm, preferably 0.2 to 50 μm, especially preferably 0.2 to 20 μm, very especially preferably 0.2 to 5 μm, in particular 0.2 to 0.8 μm, determined via Fraunhofer diffraction. The term D[4.3] refers to the volume mean diameter (see manual for Malvern Mastersizer S, Malvern Instruments Ltd., UK).

More details regarding the grinding step, and the equipment used herefor, can be found, inter alia, in Ullmann's Encyclopedia of Industrial Chemistry, Sixth Edition, 2000, Electronic Release, Size Reduction, chapter 3.6.: Wet Grinding and in EP-A-0 498 824.

In an especially preferred embodiment of the process according to the invention for the preparation of one of the abovementioned dry powders,
a) at least one active substance which is sparingly soluble, or insoluble, in water is dissolved at temperatures above 30° C. in a water-miscible, organic solvent or a mixture of water and a water-miscible, organic solvent,
b) the resulting solution is mixed with an aqueous molecularly disperse or colloid-disperse solution of a rice starch, and
c) the resulting dispersion is converted into a dry powder.

The invention also relates to pulverulent products of at least one active substance which is sparingly soluble, or insoluble, in water, which preparations are obtainable by one of the abovementioned processes.

The invention also relates to a process for the preparation of an oil-miscible product in the form of a double dispersion, comprising at least one active substance which is sparingly soluble, or insoluble, in water, wherein the aqueous dispersions described at the outset are emulsified in oil.

In this process, an emulsifier is used to form a water-in-oil emulsion in which the aqueous phase comprises protective-colloid-stabilized nanoparticles of at least one organic UV filter which is sparingly soluble, or insoluble, in water. Suitable emulsifiers are W/O emulsifiers with an HLB value of less than 10, in particular from 2 to 6, which W/O emulsifiers are known per se (cf. H. P. Fiedler, Lexikon der Hilfsstoffe für Pharmazie, Kosmetik und angrenzende Gebiete [Dictionary of adjuvants for pharmacy, cosmetics and related fields], 1996, pages 753 ff). Typical representatives of this class of emulsifier are partial esters of fatty acids with polyhydric alcohols, for example glycerol monostearate or mixtures of mono-, di- and triglycerides, partial esters of fatty acids with sorbitan and/or preferably fatty acid esters of polyglycerol, such as, for example, polyglycerol polyricinoleate, which are used in a concentration of from 10 to 1000% by weight, preferably 100 to 900% by weight, especially preferably 400 to 800% by weight, based on the active substance(s).

The dispersant can be of synthetic, mineral, vegetable or animal origin. Typical representatives are, inter alia, sesame seed oil, corn oil, cottonseed oil, soya oil or groundnut oil, esters of medium-chain fatty acids of vegetable origin, and also liquid paraffin, glyceryl stearate, isopropyl myristate, diisopropyl adipate, cetyl stearyl 2-ethylhexanoate, hydrogenated polyisobutene, Vaseline, caprylic/capric acid triglycerides, microcrystalline wax, lanolin and stearic acid. In general, the dispersant amounts to 30 to 95, preferably 50 to 80, % by weight, based on the total amount of the finished emulsion.

The emulsification can be carried out continuously or batchwise.

The physical stability of the double dispersion system, such as, for example, the sedimentation stability, is achieved by a very good fine distribution of the aqueous phase in the oil phase, for example by intensive treatment with a rotor/stator disperser at temperatures of from 20 to 80, preferably 40 to 70° C., or a high-pressure homogenizer such as an APV Gaulin apparatus or an ultra-high pressure homogenizer such as the Microfluidizer, in a pressure range of from 700 to 1000 bar. The mean diameters which can be achieved herewith, of the aqueous-disperse phase, are less than 500 μm, preferably less than 100 μm, especially preferably less than 10 μm, in particular less than 1 μm.

The invention also relates to liquid, oil-miscible products of at least one active substance which is sparingly soluble, or insoluble, in water, which products are obtainable by the abovementioned process, which, as double dispersion systems, comprise, in an oil as dispersant, an aqueous-disperse phase with a particle diameter of less than 500 μm in which protective-colloid-stabilized particles of one or more active substances which are sparingly soluble, or insoluble, in water are present in dispersed form.

The invention also relates to the use of the abovementioned aqueous dispersions as additives to foodstuffs, food supplements, animal feeds, pharmaceutical and cosmetic products.

The invention also relates to the use of the abovementioned pulverulent products as additives to foodstuffs, food supplements, animal feeds, pharmaceutical and cosmetic products.

The invention also relates to the use of the abovementioned liquid, oil-miscible products as additives to foodstuffs, food supplements, animal feeds, pharmaceutical and cosmetic products.

The invention is described in greater detail hereinbelow with reference to the examples.

### EXAMPLE 1

### Preparation of an Aqueous Astaxanthin Suspension, and Subsequent Conversion into a Dry Powder

In a vessel capable of being heated, 20 g of astaxanthin and 4 g ascorbyl palmitate were suspended in 294 g of isopropanol/water (88/12, w/w) at a temperature of 30° C. In a mixing chamber, this suspension was mixed with 536 g of isopropanol/water (88/12, w/w) at a mixing temperature of 170° C., with a residence time of 0.2 seconds. After the abovementioned residence time, the resulting molecularly disperse astaxanthin solution immediately afterwards entered a further mixing chamber in which 10.4 kg of an aqueous rice starch solution which, in addition to 108 g of rice starch, comprised 36 g of sucrose and which had been brought to pH 8 by means of sodium hydroxide solution, was admixed via a high-pressure pump in a mixing angle of 90°, during which process the astaxanthin precipitated in colloid-disperse form with a mean particle size of 144 nm at a temperature of 45° C.

The suspension was subsequently concentrated and converted in a manner known per se to give a free-flowing 10% dry astaxanthin powder with a mean particle size of 129 nm. The dry powder redissolved in water to give a clear red dispersion, the color intensity of the redispersion only falling short of that of the original dispersion by approximately 10%.

1. An aqueous dispersion comprising at least one active substance which is sparingly soluble, or insoluble, in water and rice starch as protective colloid.

2. The aqueous dispersion according to claim 1, which takes the form of a suspension comprising at least one active substance which is sparingly soluble, or insoluble, in water as nanoparticulate particles.

3. The aqueous dispersion according to claim 1, comprising 0.1 to 90% by weight of at least one active substance which is sparingly soluble, or insoluble, in water, and 0.1 to 99.9% by weight of rice starch, all percentages being based on the dry matter of the aqueous dispersion.

4. The aqueous dispersion according to claim 3, additionally comprising 0.1 to 70% by weight of at least one plasticizer, 0.01 to 70% by weight of at least one emulsifier and/or 0.01 to 50% by weight of at least one antioxidant and/or preservative.

5. A process for the preparation of an aqueous dispersion of at least one active substance which is sparingly soluble, or insoluble, in water, by dispersing, in an aqueous molecularly disperse or colloid-disperse solution of a protective colloid, one or more active substance(s) which is/are sparingly soluble, or insoluble, in water, which comprises using rice starch as protective colloid.

6. The process according to claim 5, wherein the dispersing step takes the form of the preparation of a suspension of at least one active substance which is sparingly soluble, or insoluble, in water, in an aqueous molecularly disperse or colloid-disperse solution of a rice starch.

7. The process according to claim 5, wherein the dispersing step comprises:
a₁) dissolving at least one active substance which is sparingly soluble, or insoluble, in water in one or more water-miscible organic solvents or in a mixture of water and one or more water-miscible organic solvent(s), or
a₂) dissolving, in one or more organic solvent(s) which is/are immiscible with water at least one active substance which is sparingly soluble, or insoluble, in water,
b) mixing of the solution obtained in a₁) or a₂) with an aqueous molecularly disperse or colloid-disperse solution of a rice starch, the hydrophobic phase of the active substance which is sparingly soluble, or insoluble, in water being generated as a nanodisperse phase, and
c) removing the organic solvent.

8. The process according to claim 7, wherein the organic solvent for the dispersing step is at least one water-miscible organic solvent or a mixture of water and at least one water-miscible organic solvent.

9. The process according to claim 7, wherein, the molecularly disperse solution of at least one active substance which is sparingly soluble, or insoluble, in water is prepared in step a) at temperatures above 30° C. and immediately thereafter, in step b), treated with the aqueous solution of the protective colloid, a mixing temperature of between 35° C. to 120° C. being established.

10. A process for the preparation of a dry powder comprising at least one active substance which is sparingly soluble, or insoluble, in water as nanoparticulate particles, wherein an aqueous dispersion as defined in claim 1 is freed from water and optionally dried in the presence of a coating material.

11. The process according to claim 10, wherein the aqueous dispersion is an aqueous suspension of at least one active substance which is sparingly soluble, or insoluble, in water.

12. The process according to claim 11, wherein the suspension is ground before its conversion into a dry powder.

13. The process according to claim 10, wherein
a) at least one active substance which is sparingly soluble, or insoluble, in water is dissolved at temperatures above 30° C. in a water-miscible, organic solvent or a mixture of water and a water-miscible, organic solvent,
b) the resulting solution is mixed with an aqueous molecularly disperse or colloid-disperse solution of a rice starch, and
c) the resulting dispersion is converted into a dry powder.

14. A pulverulent product of at least one active substance which is sparingly soluble, or insoluble, in water, obtainable by a process as defined in claim 10.

15. A process for the preparation of an oil-miscible product of at least one active substance which is sparingly soluble, or insoluble, in water, wherein an aqueous dispersion as defined in claim 1 is emulsified in oil in the presence of an emulsifier.

16. A liquid, oil-miscible product of at least one active substance which is sparingly soluble, or insoluble, in water, which product is obtainable by a process defined according to claim 15, wherein, as double dispersion systems, they comprise, in an oil as dispersant, an aqueous-disperse phase with a particle diameter of less than 500 μm in which protective-colloid-stabilized particles of one or more active substances which are sparingly soluble, or insoluble, in water are present in dispersed form.

17-19. (canceled)

20. Foodstuffs, food supplements, animal feeds, pharmaceutical or cosmetic products which comprise an additive and said additive is the aqueous dispersion as defined in claim 1.

21. Foodstuffs, food supplements, animal feeds, pharmaceutical or cosmetic products which comprise the pulverulent preparation as defined in claim 14.

22. Foodstuffs, food supplements, animal feeds, pharmaceutical or cosmetic products which comprise the liquid, oil-miscible preparation as defined in claim 16.
